(19) United States
(12) Patent Application Publication
(10) Pub. No.: US 2005/0266015 A1
(43) Pub. Date: Dec. 1, 2005

(54) Enhanced activity of HIV vaccine using a second generation immunomodulatory oligonucleotide

(75) Inventors: Mario Clerici, Milan (IT); Richard Bartholomew, San Diego, CA (US); Dorothy Bray, Bucks (GB); Ekambar Kandimalla, Southboro, MA (US); Sudhir Agrawal, Shrewsbury, MA (US)

Correspondence Address: KEOWN & ASSOCIATES, SUITE 1200, 500 WEST CUMMINGS PARK, WOBURN, MA, 01801, US

(73) Assignees: Hybridon, Inc.; Immune Response Corporation, Inc

(21) Appl. No.: 11/078,654

(22) Filed: Mar. 11, 2005

Related U.S. Application Data
(60) Provisional application No. 60/552,871, filed on Mar. 12, 2004.

(51) Int. Cl. A61K 39/21; A61K 48/00

(52) U.S. Cl. 424/188.1; 514/44

(57) ABSTRACT

The invention relates to the therapeutic use of a second generation immunomodulatory oligonucleotide in combination with HIV-1 antigen or immunogen to enhance the ability to reduce the risk HIV infection and to control the progression of HIV infection to prevent AIDS Related Complex (ARC) and AIDS.

### BACKGROUND OF THE INVENTION

1. Field of the Invention

The invention relates to anti-HIV applications using a second generation immunomodulatory oligonucleotide in combination with HIV antigen and/or immunogen.

2. Summary of the Related Art

Recently, several researchers have demonstrated the validity of the use of oligonucleotides as immunostimulatory agents in immunotherapy applications. The observation that phosphodiester and phosphorothioate oligonucleotides can induce immune stimulation has created interest in developing these compounds as a therapeutic tool. These efforts have focused on phosphorothioate oligonucleotides containing the natural dinucleotide CpG. Kuramoto et al., Jpn. J. Cancer Res. 83:1128-1131 (1992) teaches that phosphodiester oligonucleotides containing a palindrome that includes a CpG dinucleotide can induce interferon-alpha and gamma synthesis and enhance natural killer activity. Krieg et al., Nature 371:546-549 (1995) discloses that phosphorothioate CpG-containing oligonucleotides are immunostimulatory. Liang et al., J. Clin. Invest. 98:1119-1129 (1996) discloses that such oligonucleotides activate human B cells. Moldoveanu et al., Vaccine 16:1216-124 (1998) teaches that CpG-containing phosphorothioate oligonucleotides enhance immune response against influenza virus. McCluskie and Davis, J. Immunol. 161:4463-4466 (1998) teaches that CpG-containing oligonucleotides act as potent adjuvants, enhancing immune response against hepatitis B surface antigen. Moss et al have published CpG enhanced responses to HIV, for instance in Journal of Interferon and Cytokine Research, 20:131-1137(2000). HIV is the causative virus leading to Acquired Immune Deficiency Syndrome, also know as AIDS. AIDS has infected 60 million people since the beginning of the epidemic. Currently 40 million people are living with HIV/AIDS, 2.5 million being children.

20 million people have died of AIDS since this disease was first reported in 1981, and it has become the 4^{th} leading cause of death worldwide, accounting for 8,000 deaths per day. Attempts to develop either therapeutic or preventive vaccines have been difficult, and all have thus far failed in the clinic to show clinically relevant benefits. One therapeutic vaccine candidate, HIV-1 Immunogen, a gp120-depleted whole killed virus candidate emulsified with Incomplete Freund's Adjuvant (IFA), has been reported to induce HIV-1 specific immune responses in patients, both humoral and cell mediated. Though it does result in immune responses in a significant number of HIV infected patients, there remains a need to be able to enhance its activity through the use of immunomodulatory oligonucleotides. This need is true of all HIV vaccine candidates to date.

### BRIEF SUMMARY OF THE INVENTION

In a first embodiment, this invention provides an immunogenic composition comprising gp120 depleted HIV-1 antigen, either alone or emulsified with IFA, and an a second generation immunomodulatory oligonucleotide such as IMO1 having the structure 5′-TCTGTCRTTCT-X-TCTTRCTGTCT-5′, wherein X is a glycerol linker and R is 2′-deoxy-7-deazaguanosine.

In a second embodiment, the invention provides a method for enhancing the HIV specific immunity to HIV through use of an immunomodulatory oligonucleotide combined with HIV antigen, comprising administering to a mammal said immunogenic composition, either alone or emulsified with IFA, such as gp120-depleted HIV-1 antigen, with or without IFA or another adjuvant, and the immunomodulatory oligonucleotide having the structure 5′-TCTGTCRTTCT-X-TCTTRCTGTCT-5′ (IMO1), wherein X is a glycerol linker and R is 2′-deoxy-7-deazaguanosine. In this embodiment, the immunomodulatory oligonucleotide and HIV-1 antigen, with or without IFA, can be administered simultaneously or sequentially. In this embodiment, the HIV-1 antigen may be formulated or mixed with the immunomodulatory oligonucleotide.

In a third embodiment, the invention provides a method, as in the second embodiment, where the use of the immunomodulatory oligonucleotide combined with HIV antigen, with or without IFA prolongs the time for progression of HW infection to AIDS or prevents infection from occurring.

In a fourth embodiment, the invention provides a method for treating AIDS in a patient comprising administering HIV-1 antigen in combination with an immunomodulatory oligonucleotide such as IMO1, having the structure 5′-TCTGTCRTTCT-X-TCTTRCTGTCT-5′, wherein X is a glycerol linker and R is 2′-deoxy-7-deazaguanosine.

In a fifth embodiment, the invention provides a pharmaceutical formulation comprising HIV-1 antigen, with or without IFA, an immunomodulatory oligonucleotide having the structure 5′-TCTGTCRTTCT-X-TCTTRCTGTCT-5′, wherein X is a glycerol linker and R is 2′-deoxy-7-deazaguanosine, and a physiologically acceptable carrier.

In a sixth embodiment, the invention provides a kit comprising HIV-1 antigen, with or without IFA, and an immunomodulatory oligonucleotide having the structure 5′-TCTGTCRTTCT-X-TCTTRCTGTCT-5′, wherein X is a glycerol linker and R is 2′-deoxy-7-deazaguanosine, and wherein said kit components, when combined, produce an immunogenic composition.

### BRIEF DESCRIPTION OF THE DRAWINGS

FIGS. 1A-1F are graphical representations of the induction of IFN-γ, IL-10, RANTES, MIP-1α, MIP-1β and IL-5 in splenic mononuclear cells from mice immunized subcutaneously (s.c.) with saline or with different combinations of HIV-immunogen and IMO1.

FIG. 2 is a graphical representation of in vitro stimulated p24 and HIV-1 antigen specific IFN-γ producing lymphocytes evaluated in ELISPOT assay from mice immunized s.c. with saline or with different combinations of HIV-immunogen and IMO1.

FIG. 3 is a graphical representation of p-24-specific antibody titers in mice immunized s.c. with saline or with different combinations of HIV-immunogen and IMO1.

FIG. 4 is a graphical representation of lymphocyte proliferation responses in mice immunized s.c. with saline or with different combinations of HIV-immunogen and IMO1.

FIG. 5 is a graphical representation of IFN-γ/IL-10 ratio in mice immunized s.c. with saline or with different combinations of HIV-immunogen and IMO1. Mean values and standard errors are indicated. *=significance vs. HIV-1 immunogen alone.

FIGS. 6A-6C are graphical representations of the induction of IFN-γ, IL-10, RANTES in splenic mononuclear cells from mice immunized either s.c. or intramuscularly (i.m.) as indicated with saline or with different combinations of HIV-immunogen and IMO1.

FIG. 7 is a graphical representation of lymphocyte proliferative responses by splenic cells from mice immunized either s.c. or i.m. as indicated with saline or with different combination of HIV-1-antigen/Immunogen and IMO1. Panel A: unstimulated, HIV-1 Ag- and native p24-stimulated cells; panel B: PMA+IONO-stimulated cells. Mean values and standard errors are indicated. *=significance vs. HIV-antigen.

FIG. 8 is a graphical representation of IFN-γ ELISPOT by splenic cells: total PBMCs (panel A), CD8+ T-cells (panel B) and CD4+ T-cells (panel C) from mice immunized either s.c. or i.m as indicated with saline or with different combinations of HIV-1 Ag/Immunogen and IMO1. Mean values and standard errors are indicated. *=significance vs. HIV-1 Immunogen (i.m.).

FIG. 9 is a graphical representation of cytokine production by splenic cells from mice immunized either s.c. or i.m. as indicated; panel A: IFN-γ; panel B: IL-10, panel C: RANTES. Mean values and standard errors are indicated. *=significance vs. HIV-1 Immunogen (i.m.).

FIG. 10 is a graphical representation of cytokine production by splenic cells from mice immunized i.m. with HIV Immunogen plus IMO1 added pre- or post-emulsion. Panel A: IFN-γ; panel B: IL-10, panel C: RANTES. Mean values and standard errors are indicated. *=significance vs. post-emulsion.

### DETAILED DESCRIPTION OF THE PREFERRED EMBODIMENTS

The invention relates to the use of a second generation immunomodulatory oligonucleotide in combination with gp120 depleted HIV antigen or immunogen for enhancing protective or therapeutic HIV specific Immune responses to delay or prevent HIV infection and its subsequent progression to AIDS Related Complex (ARC) and AIDS. The issued patents, patent applications, and references that are cited herein are hereby incorporated by reference to the same extent as if each was specifically and individually indicated to be incorporated by reference. In the event of inconsistencies between any teaching of any reference cited herein and the present specification, the latter shall prevail for purposes of the invention.

The invention provides compositions and methods for enhancing the immunogenic response induced by gp120 depleted HIV-1 antigen or immunogen used for immunotherapy applications for the treatment or prevention of HIV infection. In the compositions and methods according to the invention, an immunomodulatory oligonucleotide provides an enhanced immunogenic effect when use in combination with HIV-1 antigen or HIV-1 immunogen. The virus used to produce HIV-1 antigen was an early isolate from an HIV-1 infected individual in Zaire 1976 (HZ321) and has been sequenced and contains a lade A envelope and dade G gag. This inactivated gp120-depleted UV-1 antigen is referred to as HIV-1 immunogen when it is formulated with incomplete Freund's adjuvant (IFA).

In a first embodiment, this invention provides an immunogenic composition comprising the gp120 depleted HIV-1 antigen, either alone or emulsified with IFA to yield gp120 depleted immunogen, and an immunomodulatory oligonucleotide having the structure 5′-TCTGTCRTTCT-X-TCTTRCTGTCT-5′ (IMO1), wherein X is a glycerol linker and R is 2′-deoxy-7-deazaguanosine. The immunomodulatory oligonucleotide induces an immune response when administered to a vertebrate. When used in combination with gp120 depleted HIV-1 antigen or immunogen, an enhanced therapeutic effect is obtained. In this embodiment, the gp120 depleted HIV-1 antigen or immunogen may be formulated or mixed with the immunomodulatory oligonucleotide.

In the methods according to this aspect of the invention, administration of the immunomodulatory oligonucleotide together with HIV antigen or immunogen can be by any suitable route, including, without limitation, parenteral, oral, sublingual, mucosal, transdermal, topical, intranasal, aerosol, intraocular, intratracheal, intrarectal, vaginal, by gene gun, dermal patch or in eye drop or mouthwash form. Administration of the therapeutic compositions of the immunomodulatory oligonucleotide with HIV antigen or immunogen can be carried out using known procedures at dosages and for periods of time effective to reduce symptoms or surrogate markers of the disease. When administered systemically, the therapeutic composition is preferably administered at a sufficient dosage to attain a blood level of immunomodulatory oligonucleotide from about 1 pg/mL to about 10 μg/mL. For localized administration, much lower concentrations than this may be effective, and much higher concentrations may be tolerated. Preferably, a total dosage of immunomodulatory oligonucleotide ranges from about 0.05 mg per patient per administration to about 100 mg per patient per administration while the doses of HIV immunogen and/or antigen may range from 0.05 to 0.5 mg of gp120 depleted immunogen and/or antigen per patient per administration. In further embodiments, the dose ranges are preferably from about 0.1 mg/patient to 5 mg/patient for IMO1 and 10-200 μg p24 antigen/patient administration. (Note: 10 μg p24 is equivalent to 100 μg gp120 depleted HIV-1 antigen.) In some instances it may be desirable to calculate the dose based on mg of the composition per kg of the patient's body weight per administration. It may be desirable to administer simultaneously, or sequentially a therapeutically effective amount of one or more of the therapeutic compositions of the invention to an individual as a single treatment episode.

For purposes of this aspect of the invention, the term “in combination with” means in the course of treating the same disease in the same patient, and includes administering the immunomodulatory oligonucleotide and the HIV-1 antigen in any order, including simultaneous administration, as well as any temporally spaced order, for example, from sequentially with one immediately following the other to up to several days apart. Such combination treatment may also include more than a single administration of the immunomodulatory oligonucleotide, and independently the HIV-1 antigen and/or immunogen. The administration of the immunomodulatory oligonucleotide and HIV-1 antigen or immunogen may be by the same or different routes.

The immunomodulatory oligonucleotide comprises an immunostimulatory dinucleotide of formula CpG, wherein C is cytidine; G is 2′-deoxy-7-deazaguanosine, and p is a phosphorothioate internucleoside linkage.

The immunomodulatory oligonucleotide used in the method according to the invention may conveniently be synthesized using an automated synthesizer and phosphoramidite approach. In some embodiments, the immunomodulatory oligonucleotide is synthesized by a linear synthesis approach. As used herein, the term “linear synthesis” refers to a synthesis that starts at one end of the immunomodulatory oligonucleotide and progresses linearly to the other end.

An alternative mode of synthesis for the immunomodulatory oligonucleotide is “parallel synthesis”, in which synthesis proceeds outward from a central linker moiety. A solid support attached linker can be used for parallel synthesis, as is described in U.S. Pat. No. 5,912,332. Alternatively, a universal solid support, such as phosphate attached to controlled pore glass support, can be used.

At the end of the synthesis by either linear synthesis or parallel synthesis protocols, the immunomodulatory oligonucleotide used in the methods according to the invention may conveniently be deprotected with concentrated ammonia solution or as recommended by the phosphoramidite supplier. The product immunomodulatory oligonucleotide is preferably purified by reversed phase HPLC, detritylated, desalted and dialyzed.

In a second embodiment, the invention provides a method for enhancing HIV-specific immunity aimed towards delaying progression to AIDS in patients who are infected with the virus, or for preventing infection in non-infected individuals, comprising administering to a mammal the immunogenic composition comprising gp120 depleted HIV-1 antigen or immunogen and an immunomodulatory oligonucleotide having the structure 5′-TCTGTCRTTCT-X-TCTTRCTGTCT-5′ (IMO1), wherein X is a glycerol linker and R is 2′-deoxy-7-deazaguanosine. In this embodiment, the immunomodulatory oligonucleotide and HIV-1 antigen or immunogen can be administered simultaneously or sequentially. In this embodiment, the HIV-1 antigen may be formulated or mixed with the immunomodulatory oligonucleotide.

In a third embodiment, the invention provides a method of inducing HIV-specific responses in a mammal comprising administering to a mammal the immunogenic composition comprising HIV-1 antigen or immunogen and an immunomodulatory oligonucleotide having the structure 5′-TCTGTCRTTCT-X-TCTTRCTGTCT-5′ (IMO1), wherein X is a glycerol linker and R is 2′-deoxy-7-deazaguanosine. In this embodiment, the HIV-1 antigen or immunogen may be formulated or mixed with the immunomodulatory oligonucleotide.

In a fourth embodiment, the invention provides a method for treating patients with AIDS comprising administering HIV-1 antigen or immunogen in combination with an immunomodulatory oligonucleotide having the structure 5′-TCTGTCRTTCT-X-TCTTRCTGTCT-5′ (IMO1), wherein X is a glycerol linker and R is 2′-deoxy-7-deazaguanosine. In this embodiment, the HIV-1 antigen may be formulated or mixed with the immunomodulatory oligonucleotide.

In a fifth embodiment, the invention provides a pharmaceutical formulation comprising HIV-1 antigen or immunogen, an immunomodulatory oligonucleotide having the structure 5′-TCTGTCRTTCT-X-TCTTRCTGTCT-5′ (IMO1), wherein X is a glycerol linker and R is 2′-deoxy-7-deazaguanosine, and a physiologically acceptable carrier. As used herein, the term “physiologically acceptable” refers to a material that does not interfere with the effectiveness of the immunomodulatory oligonucleotide and the HIV-1 antigen or immunogen and is compatible with a biological system such as a cell, tissue, or organism. Preferably, the biological system is a living organism, such as a vertebrate.

As used herein, the term “carrier” encompasses any excipient, diluent, filler, salt, buffer, stabilizer, solubilizer, lipid, or other material well known in the art for use in pharmaceutical formulations. It will be understood that the characteristics of the carrier, excipient, or diluent will depend on the route of administration for a particular application. The preparation of pharmaceutically acceptable formulations containing these materials is described in, e.g., Remington's Pharmaceutical Sciences, 18th Edition, ed. A. Gennaro, Mack Publishing Co., Easton, Pa., 1990.

In a sixth embodiment, the invention provides a kit comprising HIV-1 antigen or immunogen, and an immunomodulatory oligonucleotide having the structure 5′-TCTGTCRTTCT-X-TCTTRCTGTCT-5′ (IMO1), wherein X is a glycerol linker and R is 2′-deoxy-7-deazaguanosine, and wherein said kit components, when combined, produce an immunogenic composition.

The examples below are intended to further illustrate certain preferred embodiments of the invention, and are not intended to limit the scope of the invention.

### EXAMPLES

### Example 1

### Animals

Inbred, female C57BL/6 mice (from Charles River Laboratories, Calco, Italy), 6-8 weeks old, were used. Mouse colonies were maintained on a 12-h light-dark cycle in cages of 8-10 animals per group with water and food provided ad libitum.

### Example 2

### Formulations for the Animal Experiment

The IMO used in this study was provided by Hybridon, Inc. The immunomodulatory oligonucleotide IMO1, having the sequence 5′-TCTGTCRTTCT-X-TCTTRCTGTCT-5′ was utilized for the experiments. X is a glycerol linker and R is 2′-deoxy-7-deazaguanosine.

The HIV-1 antigen consists of gp120-depleted HIV-1 (HZ321; The Immune Response Corporation). Gp120-depleted HIV-1 (HZ321) antigen was highly purified by ultrafiltration and ion exchange chromatography from the extracellular supernatant of HIV-1 HZ321 Hut-78 cells. The outer envelope gp120 is depleted at the ultrafiltration stage of the purification process. Antigen preparations were inactivated through sequential application of β-propiolactone and ⁶⁰Co gamma irradiation.

### Example 3

### Protocol I Schema

Female C57/BL6 mice; 6-8 weeks of age (N=10/group) were immunized s.c. with gp120-depleted whole-killed HIV-1 immunogen (10 μg), either alone or combined with IMO1 at 10, 30 and 90 μg or mouse oligonucleotide IMO2 (30 μg) and/or gp120-depleted whole-killed HIV-1 immunogen (10g). After their primary immunization, mice were boosted with an equivalent administration 2 weeks later. On Day 28 of the study (2 weeks after the second injection), immunological analyses were carried out on fresh splenic mononuclear cells stimulated in vitro for 4 days in medium alone; with native p24 antigen; or HIV-1 antigen.

Production of IFN-gamma; IL-12; IL-5, IL-10, MIP1 alpha, MIP1 beta, RANTES was evaluated by ELISA using commercially available kits. P24 antigen- and HIV-1 antigen-specific IFN-γ-producing lymphocytes were also evaluated in ELISPOT assays. P24 antigen-; HIV-1 antigen; and LPS-specific lymphocyte proliferation was evaluated in a standard proliferation assay.

### Example 4

### Immunological Analyses

Mouse blood was collected and serum obtained was stored frozen for antibody assessments. The spleens were excised under sterile conditions in a laminar flow hood and homogenized using a Dounce homogenizer (with B pestle) for optimal cell recovery. The spleen cells were re-suspended in cell culture medium (RPMI 1640) at the desired concentration and used in culture assays.

IFN-γ, IL5, IL-10, M1P1α, M1P1β, RANTES Production Evaluated with ELISA Methods

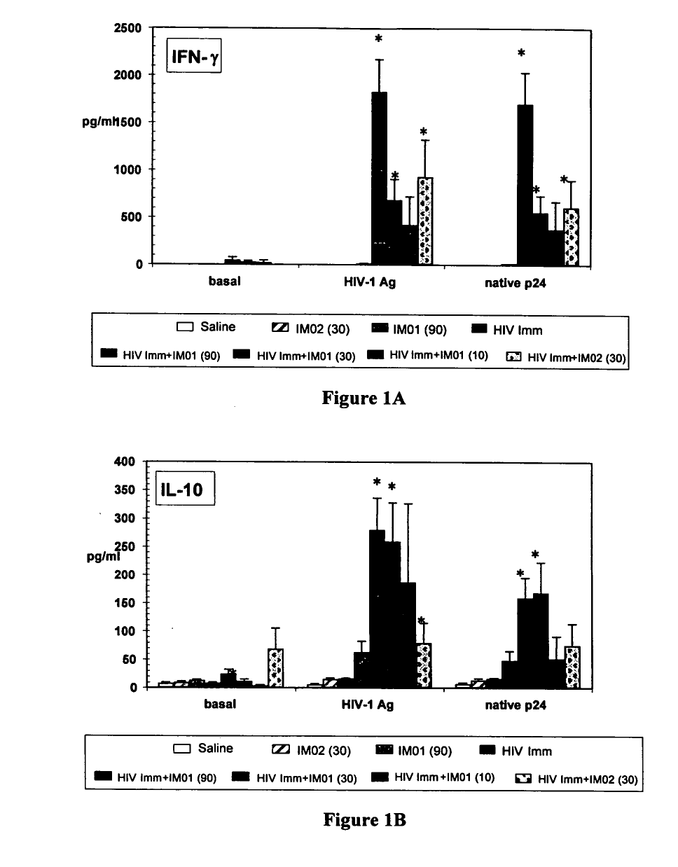

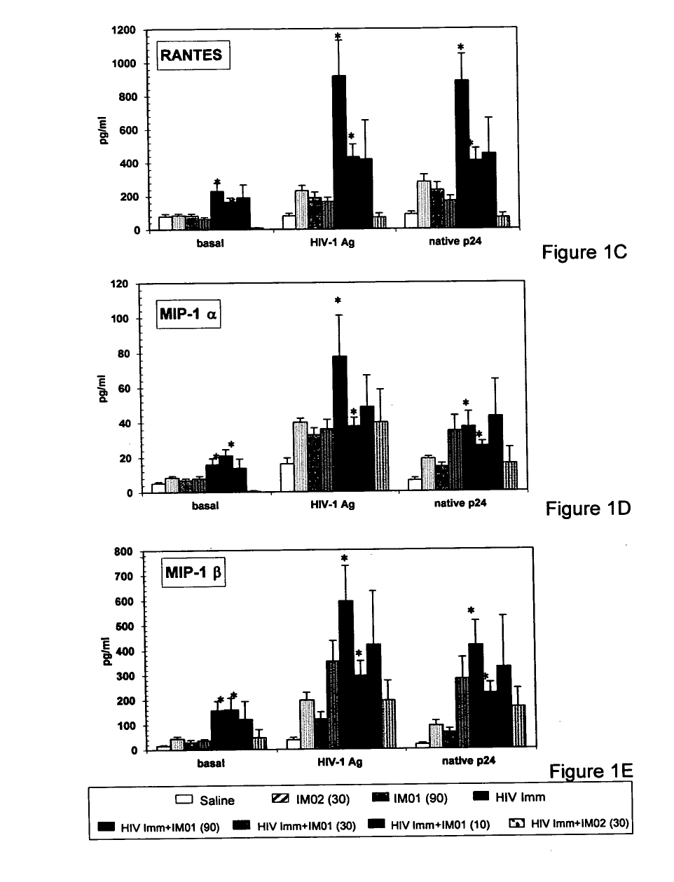

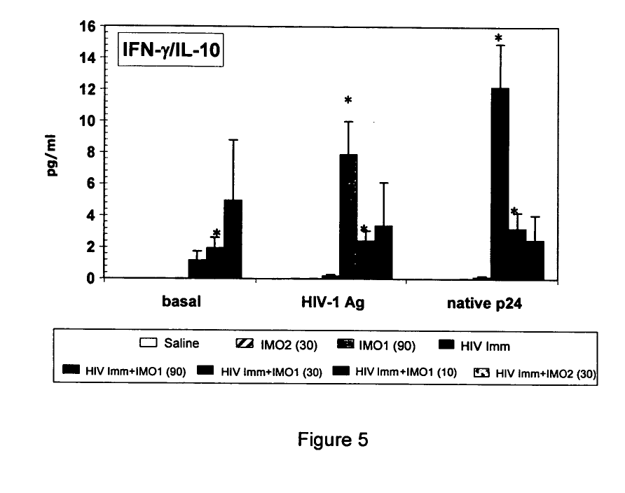

For the chemokine measurements (MIP1α, MIP1β, RANTES), fresh splenic mononuclear cells were isolated and cultured for 4 days with or without stimulation by HIV-1 antigen (10 μg/mL) or native p24 (np24) Ag (10 μg/mL) in 96-well plates in a final volume of 200 uL of RPMI 1640 medium. Supernatants were harvested and analyzed by ELISA for IFN-gamma, macrophage inflammatory protein MIP-1 alpha and beta or RANTES chemokines (R&D Systems), according to the manufacturer's recommendations. Results indicating the levels of these cytokines and chemokines following the various treatments and how they were influenced by IMO1 are shown in FIGS. 1A-1F. FIG. 5 shows that the IFN-γ/IL-10 ratio is significantly increased by IMO1, which suggests a predominant induction of IFN-γ, and stimulation of a strong cell-mediated immune response.

P24 Antigen- and HIV-1 Antigen-Specific IFN-γ-Producing Lymphocytes Evaluated in ELISPOT Assays

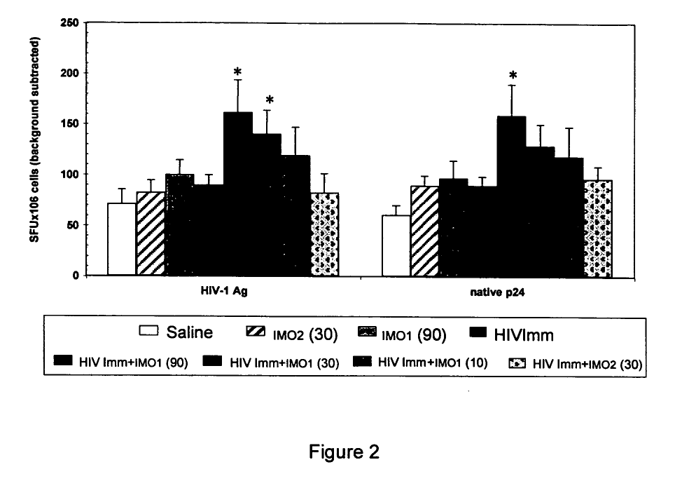

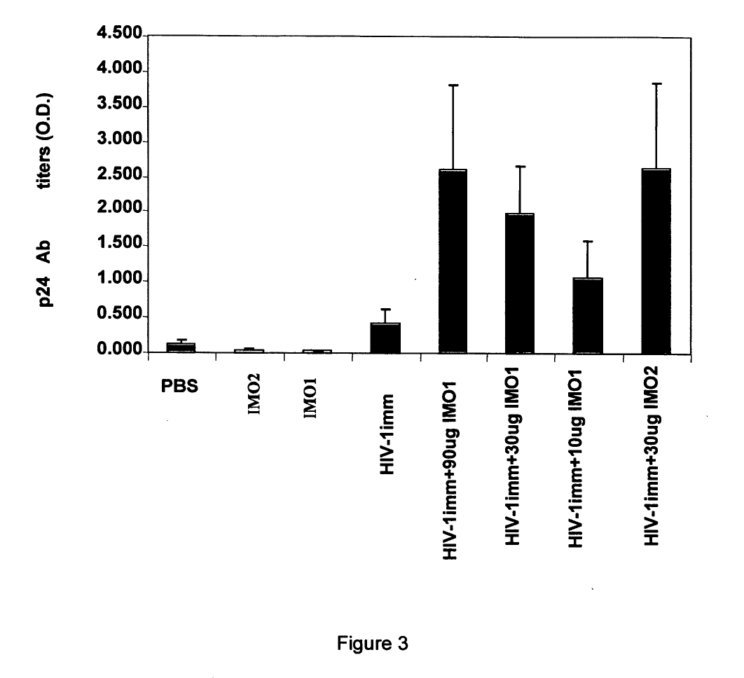

Single-cell suspensions from the spleen were prepared in PBS and plated on ninety-six well nitrocellulose plates (Millipore) that had been coated with 10 ug/mL anti-IFN-gamma (PharMingen) Ab in PBS and incubated overnight at 4° C. Plates were blocked with 10 mg/mL BSA in PBS (pH 7.4). Serially diluted (2-fold) single-cell suspensions plus supplemented RPMI 1640 medium (10% fetal calf serum) were plated at 37° C. in triplicate. Cells were left untreated or were stimulated with 5 μg/mL of the highly purified p24 (Immune Response) or with 5 ug of the highly purified HIV-1 antigen (Immune Response). After 24 h, wells were washed with PBS-Tween 20 (0 05%), and biotinylated anti-IFN-γ (PharMingen) was added to wells for 2 h at room temperature. Horseradish peroxidasestreptavidin conjugate (Sigma) was added and incubated for 1 h at room temperature, and plates were re-developed by avidin-peroxidase substrate that contained hydrogen peroxide and 3-amino-9-ethylcarboazole (Sigma) in acetate buffer. Plates were re-dried, and spots are counted using an n automated ELIspot reader. Results are shown in FIG. 2, and show IMO1 enhancement of the number of cells producing IFN-γ.

P24 Antigen-; HIV-1 Antigen; and Mitogen-Specific Lymphocyte Proliferation Evaluated in a Standard Proliferation Assay (LPAs).

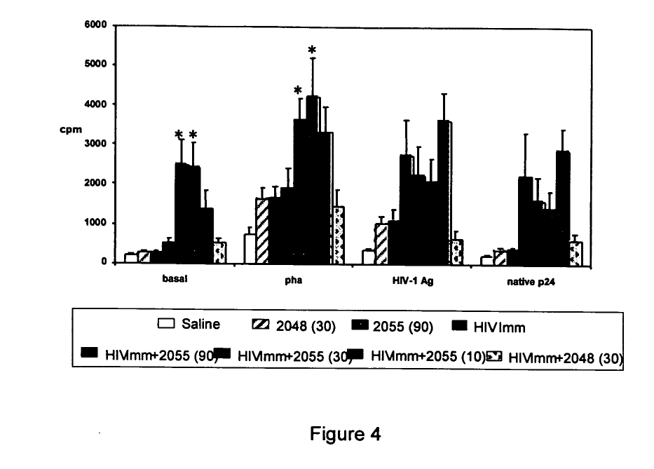

For measuring lymphocyte proliferation, fresh splenic mononuclear cells from immunized mice were purified and cultured with medium alone, PMA/ionomycin (5 ug/mL and 1 uM), or inactivated gp120-depleted HIV-1 antigen (10 ug/mL). Splenocytes were seeded in a round-bottom 96-well plate (Becton Dickinson) at 2×10⁵ cells/well in complete RPMI 1640 medium containing 10% FBS and 1% antibiotics. All assays were done in triplicate. After 5 days of incubation, cells were labeled with 1 μCi of [3H] thymidine in complete RPMI without FBS for 18 h. On day 6, cells were harvested, and the incorporated label was determined in a scintillation counter. Geometric mean counts per minute were calculated from the triplicate wells with and without stimulation by the HIV-1 antigen. Results, shown in FIG. 4, were calculated as a lymphocyte stimulation index, which is the geometric mean cpm of cells incubated with antigen divided by the geometric mean cpm of cells incubated in medium alone. Statistical analysis of the data was performed using the SPSS-PC statistical package (SPSS Inc. Chicago, Ill.). Comparisons between different groups of animals were made using a two-tailed t-test.

### Example 5

### Protocol II Schema

A second mouse experimental protocol was designed to: (1) determine if IFA was still necessary when the immunomodulatory oligonucleotide IMO1 was present in the administered dose, (2) compare s.c. and i.m. routes of injection, and (3) whether IMO1, added either before or after IFA emulsion, influenced its ability to enhance potency of HIV-1 antigen.

Female C57/BL6 mice, 6-8 weeks of age, (8 animals/group), were immunized s.c. or i.m. with 10 μg of gp120-depleted whole-killed HIV-1 immunogen and/or 90 μg IMO1. After primary immunization, mice were boosted 2 weeks later. On day 28 (2 weeks after the booster injection), HIV specific responses by immunized spleen cells were assessed as above, after in vitro stimulation with either HIV-1 antigen or native p24-antigen. Measurements included cytokine production, lymphocyte proliferation, and IFN-gamma production by ELIspot. An ELISA based assay was used to measure p24-specific antibodies in sera.

### Example 6

### Immunological Analyses

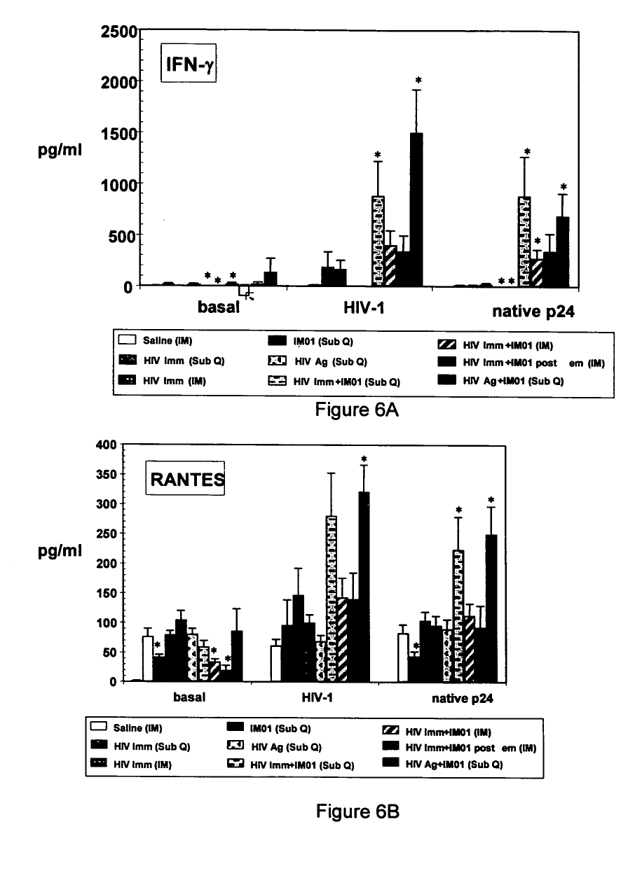

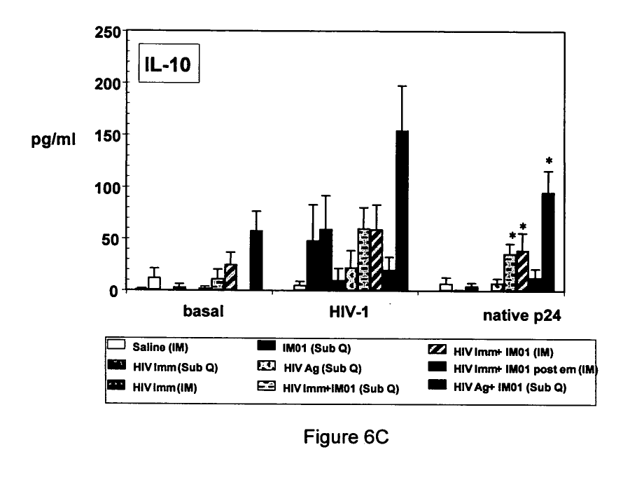

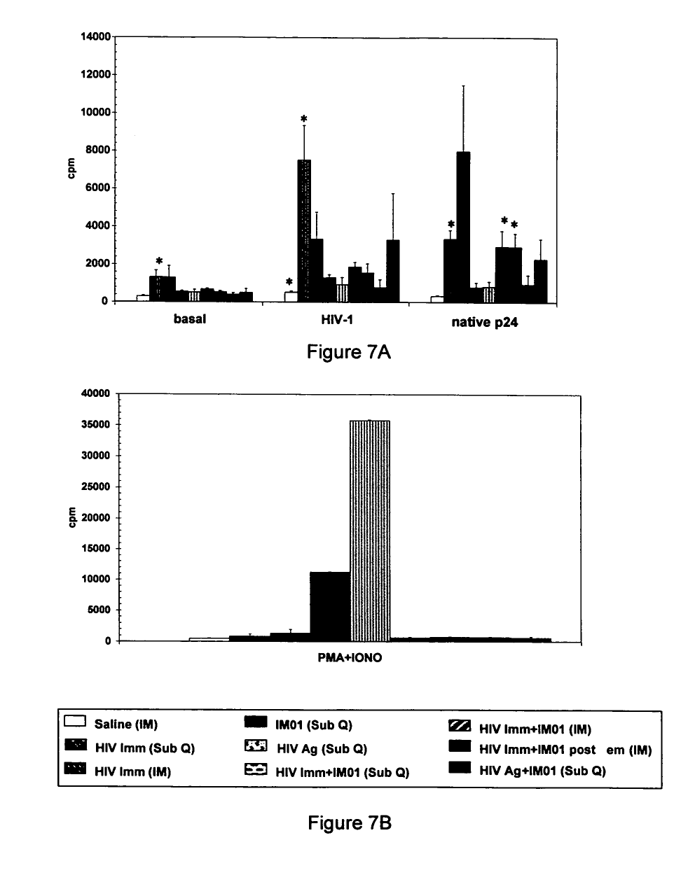

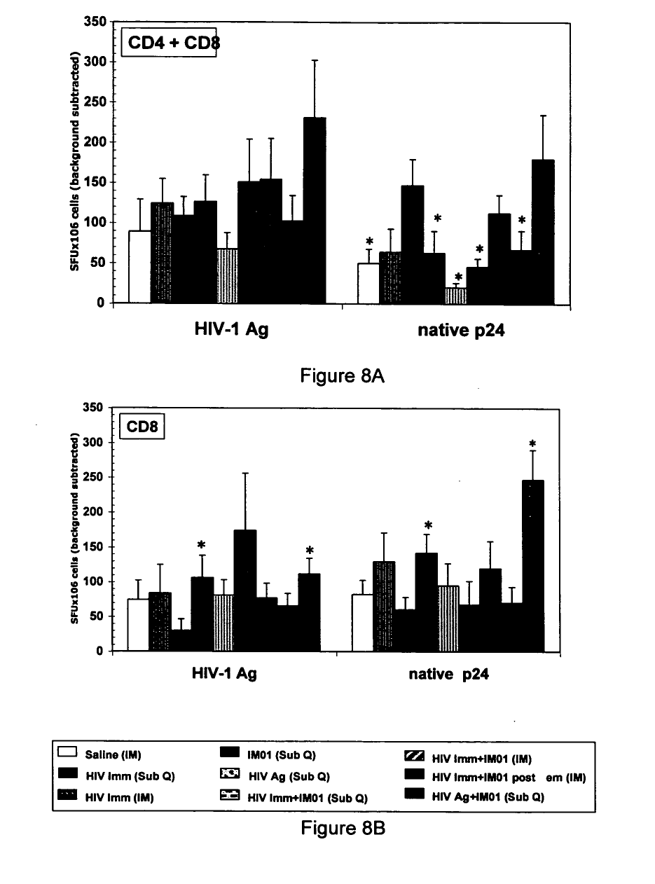

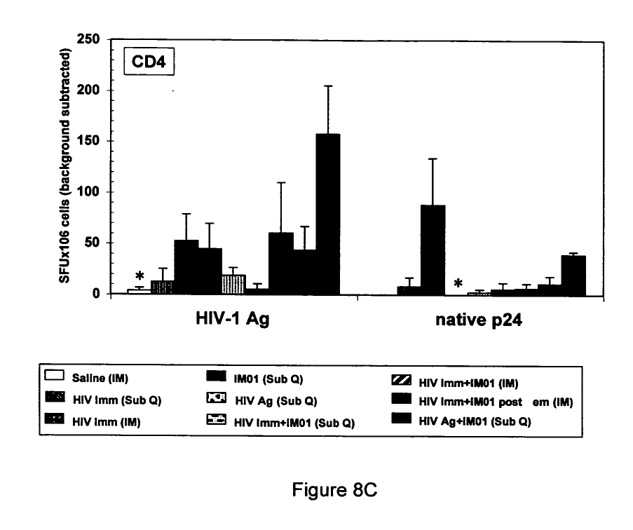

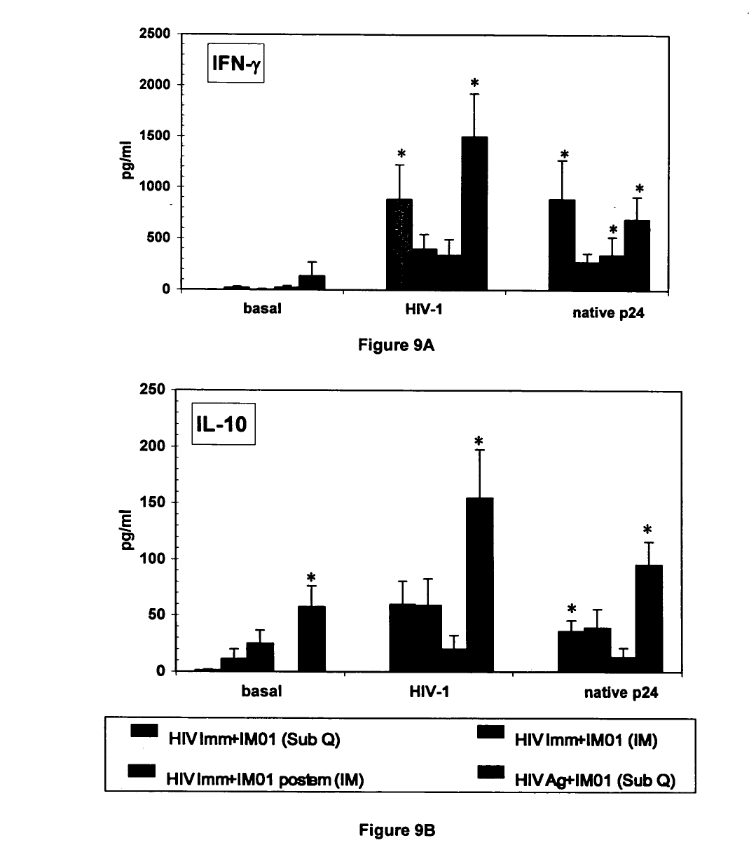

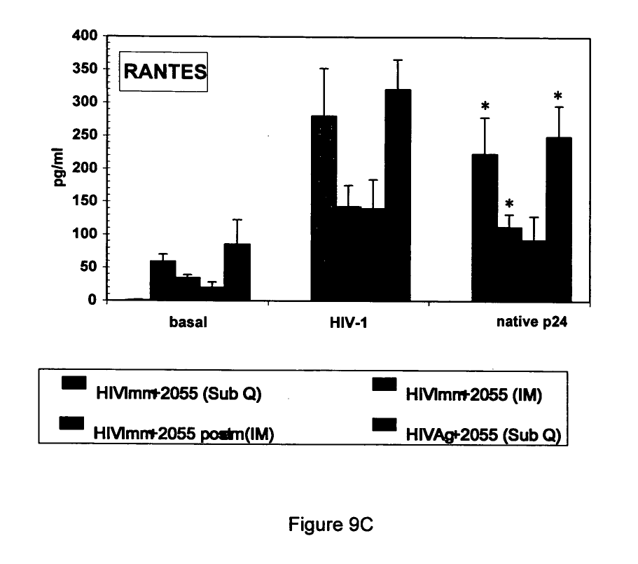

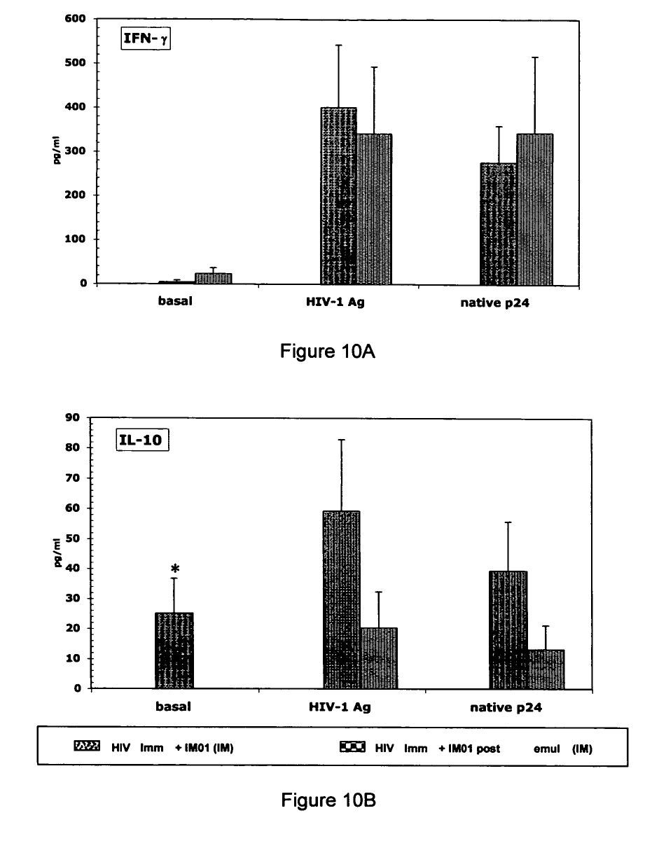

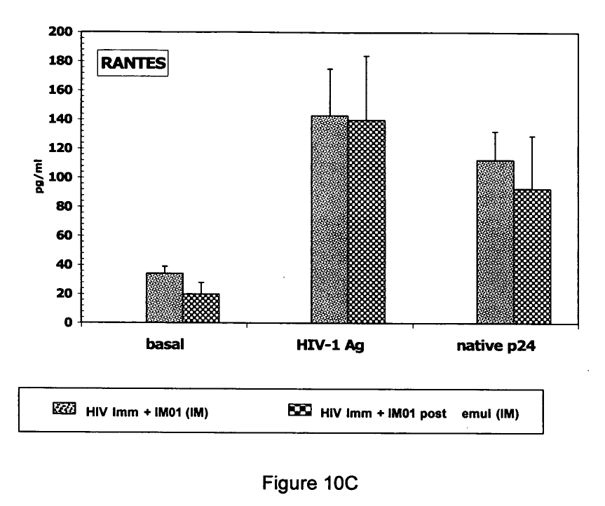

Immunological analyses were carried out as described above. Results are shown in FIGS. 6-10. Results of these experiments indicate that IMO1 significantly enhances the immunogenicity of HIV immunogen following either s.c. or i.m. administration, that the extent of enhancement is similar for formulations where IMO1 was added pre or post emulsion with IFA, and that IMO1 can enhance the immunogenicity of HIV antigen in the absence of IFA.

### Example 7

### In Vitro Effect of IMO1 on HIV Specific Immune Responses Generated by PBMCs from HIV-Infected Patients Previously Immunized with HIV-1 Immunogen

IMO1 was evaluated to determine if it could increase HIV-specific immune responses in cultures of peripheral blood mononuclear cells (PBMCs) of antiretroviral (ARV)-treated HIV patients, who were or were not immunized with HIV-immunogen (6-24 injections received every 3 months). CD4 counts, HIV plasma viremia, duration of infection, and antiretroviral therapy were comparable between the two groups of patients.

HIV-infected, highly active antiretroviral therapy (HAART)+REMUNE (inactivated gp120 depleted HIV-1 antigen emulsified with IFA)-treated patients (from Dr. Fernandez-Cruz cohort) and HIV-infected, HAART-treated patients (from the University of Milano cohort) were matched for disease duration, CD4 counts, HIV viremia, and absence/presence of protease inhibitor in their therapeutic regimen. 50 ml of whole blood was drawn by venipuncture in EDTA-containing tubes. PBMCs were stimulated in vitro with HIV-antigen, native p24, or gag in the presence of IMO1 in concentrations of: 0.1 ug/ml, 1.0 ug/ml, 10.0 ug/ml, or in medium alone.

Immunological Analyses:

p24 antigen-, HIV-1 antigen; env peptides-; gag peptides-; flu-specific IFNγ-producing CD8 lymphocytes were evaluated in ELISPOT assays (see Table 3).

TABLE 3
Elispot data from PMBCs obtained from patients receiving
HIV immunogen with IMO1 added in vitro
SFU × 10⁶ PBMC (background subtracted) CD8

| PATIENT# | | | p24 | HIV-1 | env | Gag | Flu |
|---|---|---|---|---|---|---|---|
| | Medium | | 0 | 0 | 0 | 0 | 0 |
| 1 | 0 | mg/ml IMO1 | 170 | 350 | 0 | 125 | 55 |
| 1 | 0.1 | mg/ml IMO1 | 145 | 240 | 0 | 0 | 0 |
| 1 | 1.0 | mg/ml IMO1 | 305 | 425 | 5 | 95 | 50 |
| 1 | 10 | mg/ml IMO1 | 195 | 315 | 20 | 65 | 25 |
| | Medium | | 0 | 0 | 0 | 0 | 0 |
| 2 | 0 | mg/ml IMO1 | 50 | 80 | 25 | 25 | 40 |
| 2 | 0.1 | mg/ml IMO1 | 5 | 30 | 15 | 10 | 0 |
| 2 | 1.0 | mg/ml IMO1 | 35 | 35 | 15 | 35 | 0 |
| 2 | 10 | mg/ml IMO1 | 0 | 45 | 0 | 5 | 0 |
| | Medium | | 0 | 0 | 0 | 0 | 0 |
| 3 | 0 | mg/ml IMO1 | 305 | 370 | 20 | 15 | 20 |
| 3 | 0.1 | mg/ml IMO1 | 265 | 235 | 20 | 70 | 10 |
| 3 | 1.0 | mg/ml IMO1 | 420 | 305 | 10 | 40 | 20 |
| 3 | 10 | mg/ml IMO1 | 260 | 230 | 0 | 50 | 0 |
| | Medium | | 0 | 0 | 0 | 0 | 0 |
| 4 | 0 | mg/ml IMO1 | 0 | 0 | 0 | 30 | 0 |
| 4 | 0.1 | mg/ml IMO1 | 0 | 85 | 75 | 155 | 100 |
| 4 | 1.0 | mg/ml IMO1 | 105 | 110 | 30 | 120 | 55 |
| 4 | 10 | mg/ml IMO1 | 35 | 80 | 5 | 60 | 0 |
| | Medium | | 0 | 0 | 0 | 0 | 0 |
| 5 | 0 | mg/ml IMO1 | 10 | 80 | 30 | 50 | 60 |
| 5 | 0.1 | mg/ml IMO1 | 10 | 50 | 40 | 0 | 15 |
| 5 | 1.0 | mg/ml IMO1 | 10 | 25 | 0 | 20 | 5 |
| 5 | 10 | mg/ml IMO1 | 0 | 5 | 0 | 0 | 0 |
| | Medium | | 0 | 0 | 0 | 0 | 0 |
| 6 | 0 | mg/ml IMO1 | 65 | 155 | 65 | 40 | 85 |
| 6 | 0.1 | mg/ml IMO1 | 0 | 35 | 0 | 0 | 0 |
| 6 | 1.0 | mg/ml IMO1 | 10 | 25 | 5 | 0 | 0 |
| 6 | 10 | mg/ml IMO1 | 20 | 5 | 10 | 45 | 10 |

Production of alpha-defensin was evaluated by intracellular staining in CD8+ T cells with FACS methods. The alpha-defensin results reach significance when the PBMCs are stimulated with allo-antigen (gamma irradiated peripheral blood mononuclear cells pooled from 3 different donors. (see Table 4)

TABLE 4
Defensin data from PBMCs of patients that have received multiple injection of HIV immunogen, with IMO1 added in vitro

| | | MEDIUM | | | ALLO | | | |
|---|---|---|---|---|---|---|---|---|
| | | 0 μg/ml IMO1 | 0.1 μg/ml IMO1 | 1 μg/ml IMO1 | 0 μg/ml IMO1 | 0.1 μg/ml IMO1 | 1 μg/ml IMO1 | 10 μg/ml IMO1 |
| PATIENT# | | % DEFENSIN PRODUCING CD8TCells | | | | | | |
| 1 | | 0 | 0 | 0 | 25.6 | 5.11 | 13.7 | 29.6 |
| 2 | | 0.09 | 0 | 0 | 7.52 | 0.71 | 16.2 | 14.4 |
| 3 | | 0 | 0.64 | 0.64 | 23.7 | 37.4 | 36 | 20.4 |
| 4 | | 0.1 | 0.19 | 0.14 | 5.61 | 57.3 | 26.9 | 16.7 |
| 5 | | 1.9 | 0.31 | 0.57 | 8.69 | 24.5 | 48.1 | 28.2 |
| 6 | | 1.25 | 0.4 | 0.1 | 2.56 | 45.9 | 29.2 | 42.5 |
| | MEAN | 0.556666667 | 0.256666667 | 0.241666667 | 12.28 | 28.48666667 | 28.35 | 25.3 |
| | MEDIAN | 0.625 | 0.2 | 0.05 | 14.08 | 25.505 | 21.45 | 36.05 |
| | S.D. | 0.816251595 | 0.247601831 | 0.287639821 | 9.8219326 | 22.56893942 | 12.75660613 | 10.38768502 |
| | S.E. | 0.37 | 0.11 | 0.13 | 4.38 | 10.07 | 5.7 | 4.64 |

### Equivalents

While the foregoing invention has been described in some detail for purposes of clarity and understanding, it will be appreciated by one skilled in the art from a reading of this disclosure that various changes in form and detail can be made without departing from the true scope of the invention and appended claims.

1. An HIV-1 specific immunogenic composition comprising:
a) HIV-1 antigen, either alone or admixed with IFA to yield HIV immunogen; and
b) an immunomodulatory oligonucleotide having the structure 5′-TCTGTCRTTCT-X-TCTTRCTGTCT-5′ (SEQ ID NO: 1); wherein X is a glycerol linker and R is 2′-deoxy-7-deazaguanosine.

2. An HIV-1 specific immunogenic composition comprising
a) gp120 depleted HV-1 antigen, either alone or admixed with IFA to yield HIV immunogen, and
b) an immunomodulatory oligonucleotide having the structure 5′-TCTGTCRTTCT-X-TCTTRCTGTCT-5′ (SEQ ID NO: 1) wherein X is a glycerol linker and R is 2′-deoxy-7-deazaguanosine.

3. A method for enhancing HIV specific immunity comprising administering to a mammal an immunogenic composition according to claim 1 or 2.

4. The method according to claim 3, wherein the HIV-1 antigen or HIV immunogen and the immunomodulatory oligonucleotide are administered simultaneously.

5. The method according to claim 3, wherein the HIV-1 antigen or HIV immunogen and the immunomodulatory oligonucleotide are administered sequentially.

6. The method according to claim 3, wherein the HIV-1 antigen or HIV immunogen is formulated or mixed with the immunomodulatory oligonucleotide.

7. A method for preventing HIV infection in a mammal comprising administering to the mammal an immunogenic composition comprising
a) HIV-1 antigen, either alone or admixed with IFA, and
b) an immunomodulatory oligonucleotide having the structure 5′-TCTGTCRTTCT-X-TCTTRCTGTCT-5′(SEQ ID NO: 1), wherein X is a glycerol linker and R is 2′-deoxy-7-deazaguanosine.

8. The method according to claim 7, wherein the HIV-1 antigen or HIV immunogen and the immunomodulatory oligonucleotide are administered simultaneously.

9. The method according to claim 7, wherein the HIV-1 antigen or HIV immunogen and the immunomodulatory oligonucleotide are administered sequentially.

10. The method according to claim 8, wherein the HIV-1 antigen or HIV immunogen is formulated or mixed with the immunomodulatory oligonucleotide.

11. A method for inhibiting the progression of HIV infection to AIDS comprising administering to a mammal an immunogenic composition comprising a) HIV-1 antigen or HIV immunogen, either alone or admixed with an adjuvant, and b) an immunomodulatory oligonucleotide having the structure 5′-TCTGTCRTTCT-X-TCTTRCTGTCT-5′ (SEQ ID NO: 1), wherein X is a glycerol linker and R is 2′-deoxy-7-deazaguanosine.

12. The method according to claim 11, wherein the HIV-1 antigen or immunogen and the immunomodulatory oligonucleotide are administered simultaneously.

13. The method according to claim 11, wherein the HIV-1 antigen or immunogen and the immunomodulatory oligonucleotide are administered sequentially.

14. The method according to claim 12, wherein the HIV-1 antigen or immunogen is formulated or mixed with the immunomodulatory oligonucleotide.

15. A method for treating AIDS in a mammal comprising administering to the mammal an immunogenic composition comprising a) HIV-1 antigen, either alone or admixed with an adjuvant, and b) an immunomodulatory oligonucleotide having the structure 5′-TCTGTCRTTCT-X-TCTTRCTGTCT-5′ (SEQ ID NO: 1) wherein X is a glycerol linker and R is 2′-deoxy-7-deazaguanosine.

16. The method according to claim 15, wherein the HIV-1 antigen or immunogen and the immunomodulatory oligonucleotide are administered simultaneously.

17. The method according to claim 15, wherein the HIV-1 antigen and the immunomodulatory oligonucleotide are administered sequentially.

18. The method according to claim 16, wherein the HIV-1 antigen is formulated or mixed with the immunomodulatory oligonucleotide.

19. A pharmaceutical composition comprising:
a) HIV-1 antigen, either alone or admixed with IFA;
b) an immunomodulatory oligonucleotide having the structure 5′-TCTGTCRTTCT-X-TCTTRCTGTCT-5′ (SEQ ID NO: 1); and
c) a physiologically acceptable carrier
wherein X is a glycerol linker and R is 2′-deoxy-7-deazaguanosine.

20. A kit comprising the components:
a) HIV-1 antigen, either alone or admixed with IFA; and
b) an immunomodulatory oligonucleotide having the structure 5′-TCTGTCRTTCT-X-TCTTRCTGTCT-5′ (SEQ ID NO: 1);
wherein X is a glycerol linker and R is 2′-deoxy-7-deazaguanosine, and wherein said kit components, when combined, produce an immunogenic composition.


SEQUENCE LISTING (1 sequence)

SEQ ID NO: 1 - DNA, 11 bp, Artificial Sequence
  Description of Artificial Sequence Synthetic oligonucleotide
tctgtcnttct